(19) United States
(12) Patent Application Publication
(10) Pub. No.: US 2013/0344540 A1
(43) Pub. Date: Dec. 26, 2013

(54) METHODS FOR MINIMIZING SEQUENCE SPECIFIC BIAS

(71) Applicants: Mark T. Reed, Menlo Park, CA (US); Mark Pratt, Belmont, CA (US)

(72) Inventors: Mark T. Reed, Menlo Park, CA (US); Mark Pratt, Belmont, CA (US)

(21) Appl. No.: 13/980,302

(22) PCT Filed: Jan. 10, 2012
(86) PCT No.: PCT/US12/20739

§ 371 Date: Sep. 6, 2013

Related U.S. Application Data
(60) Provisional application No. 61/439,309, filed on Feb. 3, 2011.

(51) Int. Cl. C12P 19/34

(52) U.S. Cl. CPC C12P 19/34
USPC 435/91.2

(57) ABSTRACT

Provided herein are methods of nucleic acid amplification. An exemplary method includes the steps of (a) providing a surface comprising a plurality of patches of primers, (b) providing a plurality of different nucleic acid molecules, (c) contacting the plurality of different nucleic acid molecules with the surface under conditions wherein the nucleic acid molecules bind to primers at only a subset of the patches, (d) amplifying the nucleic acid molecules under conditions to saturate the subset of patches with copies of the nucleic acid molecules, and repeating steps (c) and (d), thereby increasing the number of patches that are saturated with copies of the nucleic acid molecules.

### BACKGROUND

Amplification of template sequences by PCR typically draws on knowledge of the template sequence to be amplified such that primers can be specifically annealed to the template. The use of multiple different primer pairs to simultaneously amplify different regions of the sample is known as multiplex PCR, and suffers from limitations, including high levels of primer dimerization, and the loss of sample representation due to the different amplification efficiencies of the different regions.

For multiplex analysis of large numbers of target fragments, it is often desirable to perform a simultaneous amplification reaction for all the targets in the mixture, using a single pair of primers for all the targets. In certain embodiments, one or more of the primers may be immobilized on a solid support. Such universal amplification reactions are described more fully in application US2005/0100900 (Method of Nucleic Acid Amplification), the contents of which are incorporated herein by reference in their entirety. Isothermal amplification methods for nucleic acid amplification are described in US2008/0009420, the contents of which are incorporated herein by reference in their entirety. The methods involved may rely on the attachment of universal adapter regions, which allows amplification of all nucleic acid templates from a single pair of primers. However the universal amplification reaction can still suffer from limitations in amplification efficiency related to the sequences of the templates. One manifestation of this limitation is that the mass or size of different nucleic acid clusters varies in a sequence dependent manner. For example, AT rich clusters can gain more mass or become larger than the GC rich clusters. As a result, analysis of different clusters may lead to bias. For example, in applications where clusters are analyzed using sequencing by synthesis techniques the GC rich clusters may appear smaller or more dim such that they produce lower quality sequence data than the brighter (more intense) and larger AT rich clusters. This can result in less accurate sequence determination for the GC rich templates, an effect which may be termed GC bias. The presence of sequence specific bias during amplification gives rise to difficulties determining the sequence of certain regions of the genome, for example GC rich regions such as CpG islands in promoter regions. The resulting lack of sequence representation in the data from clusters of different GC composition translates into data analysis problems such as increases in the number of gaps in the analyzed sequence; a yield of shorter contigs, giving rise to a lower quality de-novo assembly; and a need for increased coverage to sequence a genome, thereby increasing the cost of sequencing genomes.

### SUMMARY

Provided herein is a method for amplifying nucleic acid molecules of different sequence. The method includes a first step of applying to a plurality of patches of primers immobilized on a solid surface, a first solution comprising a plurality of nucleic acid molecules of different sequence under conditions wherein one or more of the nucleic acid molecules anneals to one or more primers in a patch of primers and the annealed nucleic acid molecules are amplified until the primers in a patch are saturated to produce colonies of immobilized nucleic acid molecules, and applying to the patches of immobilized primers and colonies of the first step, a second solution comprising a plurality of nucleic acid molecules of different sequence under conditions wherein one or more of the nucleic acid molecules anneals to one or more primers in a patch of primers and the annealed nucleic acid molecules are amplified until the primers in a patch are saturated to produce colonies of immobilized nucleic acid molecules.

Also provided is a method of solid phase amplification. The method includes the steps of (a) providing a surface comprising a plurality of patches of primers, (b) providing a plurality of different nucleic acid molecules, (c) contacting the plurality of different nucleic acid molecules with the surface under conditions wherein the nucleic acid molecules bind to primers at only a subset of the patches, (d) amplifying the nucleic acid molecules under conditions to saturate the subset of patches with copies of the nucleic acid molecules, and repeating steps (c) and (d), thereby increasing the number of patches that are saturated with copies of the nucleic acid molecules.

The details of one or more embodiments are set forth in the accompanying drawings and the description below. Other features, objects, and advantages will be apparent from the description and drawings, and from the claims.

### DESCRIPTION OF DRAWINGS

FIG. 1 is a schematic showing an exemplary patterned surface with grafted primers differentially attached to exposed glass areas and blocked from interstitial areas.

FIG. 2 is a schematic showing template nucleic acid molecules seeding in isolated patches of primers.

FIG. 3 is a schematic showing GC rich template clusters grow slower than AT rich template clusters.

FIG. 4 is a schematic showing AT rich clusters saturating isolated patches of primers before GC rich clusters.

FIG. 5 is a schematic showing all clusters, whether AT or GC rich, allowed to grow or amplify until the primers in a patch are saturated.

FIGS. 6A and 6B are graphs showing Poisson statistics of repeating loading of nucleic acid molecules after six cycles.

### DETAILED DESCRIPTION

The methods and compositions presented herein are aimed at limiting the sequence specific biases found in nucleic acid amplification reactions. The methods of amplification normalize the copy number, density and signal intensity of nucleic acid clusters of different sequences. By way of example, as nucleic acid clusters expand, amplification primers on the solid support are extended, and hence adjacent clusters cannot expand over the top of each other due to the lack of available amplification primers. However, over-amplification of AT rich sequences causes rapid consumption of the primers on the surface, and hence reduces the ability of the GC rich sequences to amplify and expand. The amplification methods described herein are useful in order to obtain a high cluster density on a solid support where different clusters contain different sequences, e.g., AT and GC rich sequences.

As used herein, the term “different” when used in reference to two or more nucleic acids means that the two or more nucleic acids have nucleotide sequences that are not the same. For example, two nucleic acids can differ in the content and order of nucleotides in the sequence of one nucleic acid compared to the other nucleic acid. The term can be used to describe nucleic acids whether they are referred to as copies, amplicons, templates, targets, primers, oligonucleotides, polynucleotides or the like.

As described herein, nucleic acid templates containing a high level of A and T bases typically amplify more efficiently than nucleic acid templates with a high level of G and C bases. Nucleic acid templates with sequences containing a high level of A or T bases compared to the level of G or C bases are referred to throughout as AT rich templates or templates with high AT content. Accordingly, AT rich templates can have relatively high levels of A bases, T bases or both A and T bases. Similarly, nucleic acid templates with sequences containing a high level of G or C bases compared to the level of A or T bases are referred to throughout as GC rich templates or templates with high GC content. Accordingly, GC rich templates can have relatively high levels of G bases, C bases or both G and C bases. The terms GC rich and high GC content are used interchangeably. Similarly, the terms AT rich and high AT content are used interchangeably. The phrases GC rich and AT rich, as used herein, refer to a nucleic acid sequence having a relatively high number of G and/or C bases or A and/or T bases, respectively, in its sequence, or in a part or region of its sequence, relative to the sequence content contained within a control. In this case, the control can be similar nucleic acid sequences, genes, or the genomes from which the nucleic acid sequences originate. Generally, nucleic acid sequences having greater than about 52% GC or AT content are considered GC rich or AT rich sequences. Optionally, the GC content or AT content is greater than 55, 60, 65, 70, 75, 80, 85, 90, 95 or 99%. For example, the number of A and T bases can be at least about 10%, 25%, 50%, 75%, 100%, 2 fold, 3 fold, 4 fold or 5 fold higher than the number of G and C bases. Likewise, the number of G and C bases can be at least about 10%, 25%, 50%, 75%, 100%, 2 fold, 3 fold, 4 fold or 5 fold higher than the number of A and T bases. The methods provided herein normalize the efficiencies or levels of amplification of templates different sequence, for example, with high AT and/or GC content.

Provided herein is a method for amplifying nucleic acid molecules of different sequence. The method includes applying to a plurality of patches of primers immobilized on a solid surface, a first solution comprising a plurality of nucleic acid molecules of different sequence under conditions wherein one or more of the nucleic acid molecules anneals to one or more primers in a patch of primers and the annealed nucleic acid molecules are amplified until the primers in a patch are saturated to produce colonies of immobilized nucleic acid molecules and applying to the patches of immobilized primers and colonies, a second solution comprising a plurality of nucleic acid molecules of different sequence under conditions wherein one or more of the nucleic acid molecules anneals to one or more primers in a patch of primers and the annealed nucleic acid molecules are amplified until the primers in a patch are saturated to produce colonies of immobilized nucleic acid molecules. Optionally, the first or second solution can comprise a recombinase agent. As used throughout, the terms “first” and “second” are used for clarity purposes only and are not intended to be otherwise limiting unless specified explicitly.

As used throughout, the term “patch” refers to an area or site containing one or more primers. Each site or patch is, optionally, surrounded by an area without primers across which amplification does not occur. The sites or patches can be of any length or size and can be spaced apart from one another by any distance. Optionally, the patches are located at discrete or known positions, for example, in Cartesian or hexagonal grids.

The first solution can be applied in a first direction and the second solution can be applied in a second direction. Optionally, the solution is applied in the second direction by changing the direction of flow of the solution. The first direction can be the same as or opposite of the second direction. Likewise, the first solution can be the same as or different from the second solution. For example, solutions can be applied to a solid surface that is within the cavity of a flow cell and the solutions can flow back and forth through the flow cell and over the surface. Whether in the exemplified flow cell or other format, the first solution can be applied in a first direction followed by removal of the first solution. The second solution is then applied in the first direction or in a second direction. By way of another example, the first solution can be applied in a first direction followed by changing the flow of the first solution in the first direction to a second direction. In this example, the first solution becomes the “second” solution by changing the direction of flow of the first solution to the second direction; thus, reapplying the “first” solution to the solid surface. Optionally, after a solution is applied to a solid surface and the annealed nucleic acid molecules are amplified, the solid surface can be washed, for example, by applying a washing solution. Thus, if desired, the solid surface can be washed in between every application of solution.

The first and/or second solutions can be applied one or more times. For example, the first and/or second solutions can be applied one, two, three, four, five, six, seven, eight, nine, ten, twenty or more times. Each time a solution is applied it can be applied under conditions wherein one or more of the nucleic acid molecules of different sequence anneals to one or more primers in a patch of primers and the annealed nucleic acid molecules are amplified until the primers in a patch are saturated to produce colonies of immobilized nucleic acid molecules. Thus, in the provided methods, “solutions” can be repeatedly applied until 70, 75, 80, 85, 90, 95, 99 or 100% of the patches comprise nucleic acid molecules of different sequence (i.e., each patch is saturated with copies of a particular nucleic acid molecule).

The plurality of nucleic acid molecules can be provided in the solutions at concentrations allowing for binding of the nucleic acid molecules to one or more primers at only a subset of the patches. By way of example, the plurality of different nucleic acid molecules can be contacted with the surface to produce a Poisson distribution of occupancy in the plurality of patches that are bound to nucleic acids in the plurality of different nucleic acid molecules. Suitable concentrations of the nucleic acid molecules in a solution include, but are not limited to, 1, 2, 3, 4, 5, 6, 7, 8, 9, 10, 11, 12, 13, 14 and 15 pM. However, higher or lower concentrations of the nucleic acid molecules can be used so long as the nucleic acid molecules are provided at concentrations allowing for binding of the nucleic acid molecules to one or more primers at only a subset of the patches.

Each time the solution is applied nucleic acid molecules can bind to primers at approximately 5, 10, 15, 20, 25, 30, 35, 40, 45 or 50% of the patches. Optionally, the first time the solution is applied nucleic acid molecules can bind to primers at approximately 35% of the patches. The second time the solution is applied nucleic acid molecules can bind to primers at approximately 20% of the remaining patches. The conditions under which the plurality of different nucleic acid molecules is contacted with the surface generally produces no more than 50% of the patches in the plurality of patches that are bound to a nucleic acid in the plurality of different nucleic acid molecules. Optionally, each time the solution is applied approximately one nucleic acid molecule binds to one patch of primers.

The concentration of the plurality of nucleic acid molecules can be reduced by 1, 5, 10, 15, 20 or 25% each time a solution is applied to the solid surface. Optionally, after the first time the solution is applied to the solid surface, each subsequent time the solution is applied the concentration of the plurality of nucleic acid molecules in the next application step can be, optionally, 1, 5, 10, 15, 20 or 25% less than the concentration of the plurality of nucleic acid molecules in the prior application step. The provided methods, optionally, further comprise a finishing step wherein the concentration of the plurality of nucleic acid molecules of different sequence in the finishing step is higher than the concentration of the plurality of nucleic acid molecules of different sequence in the previous application step.

Also provided is a method of solid phase amplification. The method includes providing a surface comprising a plurality of patches of primers, providing a plurality of different nucleic acid molecules, contacting the plurality of different nucleic acid molecules with the surface under conditions wherein the nucleic acid molecules bind to primers at only a subset of the patches, amplifying the nucleic acid molecules under conditions to saturate the subset of patches with copies of the nucleic acid molecules, and repeating these steps, thereby increasing the number of patches that are saturated with copies of the nucleic acid molecules. The conditions under which the plurality of different nucleic acid molecules is contacted with the surface generally produces no more than 50% of the patches in the plurality of patches that are bound to a nucleic acid in the plurality of different nucleic acid molecules. Optionally, the conditions under which the plurality of different nucleic acid molecules is contacted with the surface can produce a Poisson distribution of occupancy in the plurality of patches that are bound to nucleic acids in the plurality of different nucleic acid molecules.

As described in more detail throughout, the same or different primer sequences can be present at the patches in the plurality of patches. Optionally, the nucleic acid molecules in the plurality of different nucleic acid molecules comprise common primer binding sequences flanking different target sequences.

In the provided methods described throughout, the plurality of different nucleic acid molecules can comprise nucleic acid molecules having AT rich sequences and nucleic acid molecules having GC rich sequences. Optionally, the subset of patches that saturate with copies of nucleic acid molecules comprise patches having copies of the AT rich sequences and patches having copies of the GC rich sequences. Optionally, the patches having copies of the AT rich sequences are of approximately equal density, approximately equal copy number, or approximately equal signal intensity as the patches having copies of the GC rich sequences. Accordingly, when detected, for example, using optical means, the AT rich clusters that are made using the methods provided herein will appear to have a similar intensity as the GC rich clusters.

As described herein, after nucleic acid molecules are annealed to the primers, the annealed nucleic acid molecules are amplified until the primers in a patch are saturated. As used herein, the term saturated refers to the occupancy of the patch of primers. When a patch is saturated, the patch is occupied with amplified nucleic acid molecules to an extent that (1) attachment of a second nucleic acid molecule is prevented or (2) a second nucleic acid molecule may be attached, but is unable to amplify or (3) a second or invading nucleic acid molecule cannot amplify to significant numbers relative to the amplified nucleic acid molecules. For example, a patch can be saturated when 50, 55, 60, 65, 70, 75, 80, 85, 90, 95, 99% or more of patch is occupied with amplified nucleic acid molecules. If present, the second or invading nucleic acid molecule typically is less than 1, 0.5, 0.25, 0.1, 0.001 or 0.0001% of the total population of nucleic acid molecules in a patch. Thus, the second or invading nucleic acid molecule, if present, cannot be optically detected or detection of the second or invading nucleic acid molecule is considered background noise or does not interfere with the detection of the originally immobilized and amplified nucleic acid sequences in the patch. In such embodiments, the patch will be apparently homogeneous or uniform in accordance with the resolution of the methods or apparatus used to detect the nucleic acid molecules in the patch. The term “saturated” as used herein is not meant to imply that every primer in a patch is used in an amplification reaction (i.e., is extended to form immobilized nucleic acid molecules). Thus, the term saturated includes conditions wherein less than 20, 15, 10 or 5% of the primers in a patch comprising immobilized nucleic acid molecules are free. In other words, in a saturated colony of immobilized nucleic acid molecules, 0.5, 1, 2, 3, 4, 5, 6, 7, 8, 9, 10, 12, 15, 17, 20% or more of the primers can be free (i.e., the primers have not been extended to form an immobilized nucleic acid molecule).

A surface or support for use in the provided methods described herein refers to any surface or collection of surfaces to which nucleic acids can be attached. Suitable surfaces include, but are not limited to, beads, resins, gels, wells, columns, chips, flowcells, membranes, matrices, plates or filters. For example, the surface can be latex or dextran beads, polystyrene or polypropylene surfaces, polyacrylamide gels, gold surfaces, glass surfaces, optical fibers, or silicon wafers. The surface can be any material that amenable to chemical modification to afford covalent linkage to a nucleic acid. Optionally, the solid surface can be a bead and wherein one patch of primers is located on one bead.

Optionally, the surface is contained in a vessel or chamber such as a flow cell, allowing convenient movement of liquids across the surface to enable the transfer of reagents. Exemplary flow cells that can be used in this manner are described in WO 2007/123744, which is incorporated herein by reference in its entirety. Optionally, the flowcell is a patterned flowcell. Suitable patterned flowcells include, but are not limited to, flowcells described in WO 2008/157640, which is incorporated by reference herein in its entirety.

Optionally, the surface may comprise a layer or coating of a material with reactive groups permitting attachment of polynucleotides. The polynucleotides are then attached to the material (e.g., covalently), which is attached to the surface (e.g., noncovalently). Such a surface is described in WO 05/65814, which is incorporated by reference herein in its entirety.

The term “immobilized” as used herein is intended to encompass direct or indirect attachment to a solid support via covalent or non-covalent bond(s). In particular embodiments, all that is required is that the molecules (for example, nucleic acids) remain immobilized or attached to a support under conditions in which it is intended to use the support, for example in applications requiring nucleic acid amplification and/or sequencing. For example, oligonucleotides or primers are immobilized such that a 3′ end is available for enzymatic extension and/or at least a portion of the sequence is capable of hybridizing to a complementary sequence. Immobilization can occur via hybridization to a surface attached primer, in which case the immobilized primer or oligonucleotide may be in the 3′-5′ orientation. Alternatively, immobilization can occur by means other than base-pairing hybridization, such as the covalent attachment.

As used throughout, nucleic acid molecules include deoxyribonucleic acids (DNA), ribonucleic acids (RNA) or other form of nucleic acid. The polynucleotide molecule can be any form of natural, synthetic or modified DNA, including, but not limited to, genomic DNA, copy DNA, complementary DNA, or recombinant DNA. Alternatively, the polynucleotide molecule can be any form of natural, synthetic or modified RNA, including, but not limited to mRNA, ribosomal RNA, microRNA, siRNA or small nucleolar RNA. The polynucleotide molecule can be partially or completely in double-stranded or single-stranded form. The terms “nucleic acid,” “nucleic acid molecule,” “oligonucleotide,” and “polynucleotide” are used interchangeably throughout. The different terms are not intended to denote any particular difference in size, sequence, or other property unless specifically indicated otherwise. For clarity of description the terms may be used to distinguish one species of molecule from another when describing a particular method or composition that includes several molecular species.

Nucleic acid molecules for use in the provided methods may be obtained from any biological sample using known, routine methods. Suitable biological samples include, but are not limited to, a blood sample, biopsy specimen, tissue explant, organ culture, biological fluid or any other tissue or cell preparation, or fraction or derivative thereof or isolated therefrom. The biological sample can be a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, stem cells, germ cells (e.g. sperm, oocytes), transformed cell lines and the like. For example, polynucleotide molecules may be obtained from primary cells, cell lines, freshly isolated cells or tissues, frozen cells or tissues, paraffin embedded cells or tissues, fixed cells or tissues, and/or laser dissected cells or tissues. Biological samples can be obtained from any subject or biological source including, for example, human or non-human animals, including mammals and non-mammals, vertebrates and invertebrates, and may also be any multicellular organism or single-celled organism such as a eukaryotic (including plants and algae) or prokaryotic organism, archaeon, microorganisms (e.g. bacteria, archaea, fungi, protists, viruses), and aquatic plankton.

Once the nucleic acid molecules are obtained, the plurality of nucleic acid molecules of different sequence for use in the provided methods may be prepared using a variety of standard techniques available and known. Exemplary methods of polynucleotide molecule preparation include, but are not limited to, those described in Bentley et al., Nature 456:49-51 (2008); U.S. Pat. No. 7,115,400; and U.S. Patent Application Publication Nos. 2007/0128624; 2009/0226975; 2005/0100900; 2005/0059048; 2007/0110638; and 2007/0128624, each of which is herein incorporated by reference in its entirety. For example, nucleic acid molecules comprise one or more regions of known sequence (e.g., an adaptor) located on the 5′ and/or 3′ ends. When the nucleic acid molecules comprise known sequences on the 5′ and 3′ ends, the known sequences can be the same or different sequences. Optionally, a known sequence located on the 5′ and/or 3′ ends of the polynucleotide molecules is capable of hybridizing to one or more primers immobilized on the surface. For example, a nucleic acid molecule comprising a 5′ known sequence may hybridize to a first plurality of primers while the 3′ known sequence may hybridize to a second plurality of primers. Optionally, nucleic acid molecules comprise one or more detectable labels. The one or more detectable labels may be attached to the nucleic acid template at the 5′ end, at the 3′ end, and/or at any nucleotide position within the nucleic acid molecule. The nucleic acid molecules for use in the provide methods comprise the nucleic acid to be amplified and/or sequenced and, optionally, short nucleic acid sequences at the 5′ and/or 3′ end(s).

A short nucleic acid sequence that is added to the 5′ and/or 3′ end of a nucleic acid molecule can be a universal sequence. A universal sequence is a region of nucleotide sequence that is common to, i.e., shared by, two or more nucleic acid molecules, where the two or more nucleic acid molecules also have regions of sequence differences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize specifically to such a universal sequence. The nucleic acid molecules may be modified to attach universal adapters (e.g., non-target nucleic acid sequences) to one or both ends of the different target sequences, the adapters providing sites for hybridization of universal primers. This approach has the advantage that it is not necessary to design a specific pair of primers for each nucleic acid molecule to be generated, amplified, sequenced, and/or otherwise analyzed; a single pair of primers can be used for amplification of different nucleic acid molecule provided that each nucleic acid molecule is modified by addition of the same universal primer-binding sequences to its 5′ and 3′ ends.

The nucleic acid molecules can also be modified to include any nucleic acid sequence desirable using standard, known methods. Such additional sequences may include, for example, restriction enzyme sites, or indexing tags in order to permit identification of amplification products of a given nucleic acid sequence.

“Primer oligonucleotides”, “oligonucleotide primers” and “primers” are used throughout interchangeably and are polynucleotide sequences that are capable of annealing specifically to one or more nucleic acid molecule templates to be amplified. Generally primer oligonucleotides are single stranded or partially single stranded. Primers may also contain a mixture of non-natural bases, non-nucleotide chemical modifications or non-natural backbone linkages so long as the non-natural entities do not interfere with the function of the primer. Optionally, a patch of primers can comprises one or more different pluralities of primer molecules. By way of example, a patch can comprise a first, second, third, fourth, or more pluralities of primer molecules each plurality having a different sequence. It will be understood that for embodiments having different pluralities of primers in a single patch, the different pluralities of primers can share a common sequence so long as there is a sequence difference between at least a portion of the different pluralities. For example, a first plurality of primers can share a sequence with a second plurality of primers as long the primers in one plurality have a different sequence not found in the primers of the other plurality.

The nucleic acid molecules are typically attached to the surface by hybridization or annealing to one or more primers in a patch of primers. Hybridization is accomplished, for example, by ligating an adapter to the ends of the nucleic acid molecules. The nucleic acid sequence of the adapter can be complementary to the nucleic acid sequence of the primer, thus, allowing the adapter to bind or hybridize to the primer on the surface. Optionally, the nucleic acid molecules are single or double stranded and adapters are added to the 5′ and/or 3′ ends of the nucleic acid molecules. Optionally, the nucleic acid molecules are double-stranded and adapters are ligated onto the 3′ ends of double-stranded nucleic acid molecule. Optionally, nucleic acid molecules are used without any adapter. In some embodiments nucleic acid molecules can be attached to a surface by interactions other than hybridization to a complementary primer. For example, a nucleic acid can be covalently attached to a surface using a chemical linkage such as those resulting from click chemistry or a receptor-ligand interaction such as streptavidin-biotin binding.

Nucleic acid amplification includes the process of amplifying or increasing the numbers of a nucleic acid molecule template and/or of a complement thereof that are present, by producing one or more copies of the template and/or or its complement. In the provided methods, amplification can be carried out by a variety of known methods under conditions including, but not limited to, thermocycling amplification or isothermal amplification. For example, methods for carrying out amplification are described in U.S. Publication No. 2009/0226975; WO 98/44151; WO 00/18957; WO 02/46456; WO 06/064199; and WO 07/010251; which are incorporated by reference herein in their entireties. Briefly, in the provided methods, amplification can occur on the surface to which the polynucleotide molecules are attached. This type of amplification can be referred to as solid phase amplification, which when used in reference to nucleic acids, refers to any nucleic acid amplification reaction carried out on or in association with a surface (e.g., a solid support). Typically, all or a portion of the amplified products are synthesized by extension of an immobilized primer. Solid phase amplification reactions are analogous to standard solution phase amplifications except that at least one of the amplification primers is immobilized on a surface (e.g., a solid support).

Suitable conditions include providing appropriate buffers/solutions for amplifying nucleic acid molecules. Such solutions include, for example, an enzyme with polymerase activity, nucleotide triphosphates, and, optionally, additives such as DMSO or betaine. Optionally, amplification is carried out in the presence of a recombinase agent as described in U.S. Pat. No. 7,485,428, which is incorporated by reference herein in its entirety, which allows for amplification without thermal melting. Briefly, recombinase agents such as the RecA protein from E. coli (or a RecA relative from other phyla), in the presence of, for example, ATP, dATP, ddATP, UTP, or ATPyS, will form a nucleoprotein filament around single-stranded DNA (e.g., a primer). When this complex comes in contact with homologous sequences the recombinase agent will catalyze a strand invasion reaction and pairing of the primer with the homologous strand of the target DNA. The original pairing strand is displaced by strand invasion leaving a bubble of single stranded DNA in the region, which serves as a template for amplification.

Solid-phase amplification may comprise a nucleic acid amplification reaction comprising only one species of oligonucleotide primer immobilized to a surface. Alternatively, as discussed above, the surface may comprise a plurality of first and second different immobilized oligonucleotide primer species. Solid phase nucleic acid amplification reactions generally comprise at least one of two different types of nucleic acid amplification, interfacial and surface (or bridge) amplification. For instance, in interfacial amplification the solid support comprises a template nucleic acid molecule that is indirectly immobilized to the solid support by hybridization to an immobilized oligonucleotide primer, the immobilized primer may be extended in the course of a polymerase-catalyzed, template-directed elongation reaction (e.g., primer extension) to generate an immobilized nucleic acid molecule that remains attached to the solid support. After the extension phase, the nucleic acids (e.g., template and its complementary product) are denatured such that the template nucleic acid molecule is released into solution and made available for hybridization to another immobilized oligonucleotide primer. The template nucleic acid molecule may be made available in 1, 2, 3, 4, 5 or more rounds of primer extension or may be washed out of the reaction after 1, 2, 3, 4, 5 or more rounds of primer extension.

In surface (or bridge) amplification, an immobilized nucleic acid molecule hybridizes to an immobilized oligonucleotide primer. The 3′ end of the immobilized nucleic acid molecule provides the template for a polymerase-catalyzed, template-directed elongation reaction (e.g., primer extension) extending from the immobilized oligonucleotide primer. The resulting double-stranded product “bridges” the two primers and both strands are covalently attached to the support. In the next cycle, following denaturation that yields a pair of single strands (the immobilized template and the extended-primer product) immobilized to the solid support, both immobilized strands can serve as templates for new primer extension.

As described throughout, the provided methods can be used to produce colonies of immobilized nucleic acid molecules. For example, the methods can produce clustered arrays of nucleic acid colonies, analogous to those described in U.S. Pat. No. 7,115,400; U.S. Publication No. 2005/0100900; WO 00/18957; and WO 98/44151, which are incorporated by reference herein in their entireties. “Clusters” and “colonies” are used interchangeably and refer to a plurality of copies of a nucleic acid sequence and/or complements thereof attached to a surface. Typically, the cluster comprises a plurality of copies of a nucleic acid sequence and/or complements thereof, attached via their 5′ termini to the surface. The copies of nucleic acid sequences making up the clusters may be in a single or double stranded form.

Each colony can comprise nucleic acid molecules of the same sequences. In particular embodiments, the sequence of the nucleic acid molecules of one colony is different from the sequence of the nucleic acid molecules of another colony. Thus, each colony comprises a different nucleic acid sequence. All of the immobilized nucleic acid molecules in a colony are typically produced by amplification of the same nucleic acid molecule. In some embodiments it is possible that a colony of immobilized nucleic acid molecules contains one or more primers without an immobilized nucleic acid molecule to which another nucleic acid molecule of different sequence can bind upon additional application of solutions containing free or unbound nucleic acid molecules. However, due to the lack of sufficient numbers of free primers in a colony, this second or invading nucleic acid molecule cannot amplify to significant numbers. The second or invading nucleic acid molecule typically is less than 1, 0.5, 0.25, 0.1, 0.001 or 0.0001% of the total population of nucleic acid molecules in a single colony. Thus, the second or invading nucleic acid molecule cannot be optically detected or detection of the second or invading nucleic acid molecule is considered background noise or does not interfere with detection of the original, immobilized nucleic acid sequences in the colony. In such embodiments, the colony will be apparently homogeneous or uniform in accordance with the resolution of the methods or apparatus used to detect the colony.

The clusters can have different shapes, sizes and densities depending on the conditions used. For example, clusters can have a shape that is substantially round, multi-sided, donut-shaped or ring-shaped. The diameter or maximum cross section of a cluster can be from about 0.2 μm to about 6 μm, about 0.3 μm to about 4 μm, about 0.4 μm to about 3 μm, about 0.5 μm to about 2 μm, about 0.75 μm to about 1.5 μm, or any intervening diameter. Optionally, the diameter or maximum cross section of a cluster can be at least about 0.5 μm, at least about 1 μm, at least about 1.5 μm, at least about 2 μm, at least about 2.5 μm, at least about 3 μm, at least about 4 μm, at least about 5 μm, or at least about 6 μm. The diameter of a cluster may be influenced by a number of parameters including, but not limited to, the number of amplification cycles performed in producing the cluster, the length of the nucleic acid template, the GC content of the nucleic acid template, the shape of a patch to which the primers are attached, or the density of primers attached to the surface upon which clusters are formed. However, as discussed above, in all cases, the diameter of a cluster can be no larger than the patch upon which the cluster is formed. For example, if a patch is a bead, the cluster size will be no larger than the surface area of the bead. The density of clusters can be in the range of at least about 0.1/mm², at least about 1/mm², at least about 10/mm², at least about 100/mm², at least about 1,000/mm², at least about 10,000/mm² to at least about 100,000/mm². Optionally, the clusters have a density of, for example, 100,000/mm² to 1,000,000/mm² or 1,000,000/mm² to 10,000,000/mm². The methods provided herein can produce colonies that are of approximately equal size. This occurs regardless of the differences in efficiencies of amplification of the nucleic acid molecules of different sequence.

Clusters may be detected, for example, using a suitable imaging means, such as, a confocal imaging device or a charge coupled device (CCD) or CMOS camera. Exemplary imaging devices include, but are not limited to, those described in U.S. Pat. Nos. 7,329,860; 5,754,291; and 5,981,956; and WO 2007/123744, each of which is herein incorporated by reference in its entirety. The imaging means may be used to determine a reference position in a cluster or in a plurality of clusters on the surface, such as the location, boundary, diameter, area, shape, overlap and/or center of one or a plurality of clusters (and/or of a detectable signal originating therefrom). Such a reference position may be recorded, documented, annotated, converted into an interpretable signal, or the like, to yield meaningful information.

Optionally, the nucleic acid molecules in the colonies can be sequenced. The sequencing is carried out by a variety of known methods, including, but not limited to, sequencing by ligation, sequencing by synthesis or sequencing by hybridization.

Sequencing by synthesis, for example, is a technique wherein nucleotides are added successively to a free 3′ hydroxyl group, typically provided by annealing of an oligonucleotide primer (e.g., a sequencing primer), resulting in synthesis of a nucleic acid chain in the 5′ to 3′ direction. These and other sequencing reactions may be conducted on the herein described surfaces bearing nucleic acid clusters. The reactions comprise one or a plurality of sequencing steps, each step comprising determining the nucleotide incorporated into a nucleic acid chain and identifying the position of the incorporated nucleotide on the surface. The nucleotides incorporated into the nucleic acid chain may be described as sequencing nucleotides and may comprise one or more detectable labels. Suitable detectable labels, include, but are not limited to, haptens, radionucleotides, enzymes, fluorescent labels, chemiluminescent labels, and/or chromogenic agents. One method for detecting fluorescently labeled nucleotides comprises using laser light of a wavelength specific for the labeled nucleotides, or the use of other suitable sources of illumination. The fluorescence from the label on the nucleotide may be detected by a CCD camera or other suitable detection means. Suitable instrumentation for recording images of clustered arrays is described in WO 07/123744, the contents of which are incorporated herein by reference herein in its entirety.

Optionally, cycle sequencing is accomplished by stepwise addition of reversible terminator nucleotides containing, for example, a cleavable or photobleachable dye label as described, for example, in U.S. Pat. No. 7,427,673; U.S. Pat. No. 7,414,116; WO 04/018497; WO 91/06678; WO 07/123744; and U.S. Pat. No. 7,057,026, the disclosures of which are incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Alternatively, pyrosequencing techniques may be employed. Pyrosequencing detects the release of inorganic pyrophosphate (PPi) as particular nucleotides are incorporated into the nascent strand (Ronaghi et al., (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568; and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

Additional exemplary sequencing-by-synthesis methods that can be used with the methods described herein include those described in U.S. Patent Publication Nos. 2007/0166705; 2006/0188901; 2006/0240439; 2006/0281109; 2005/0100900; U.S. Pat. No. 7057026; WO 05/065814; WO 06/064199; WO 07/010251, the disclosures of which are incorporated herein by reference in their entireties.

Alternatively, sequencing by ligation techniques are used. Such techniques use DNA ligase to incorporate oligonucleotides and identify the incorporation of such oligonucleotides and are described in U.S. Pat. No 6,969,488; U.S. Pat. No. 6,172,218; and U.S. Pat. No. 6,306,597; the disclosures of which are incorporated herein by reference in their entireties. Other suitable alternative techniques include, for example, fluorescent in situ sequencing (FISSEQ), and Massively Parallel Signature Sequencing (MPSS).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a method is disclosed and discussed and a number of modifications that can be made to the method steps are discussed, each and every combination and permutation of the method steps, and the modifications that are possible are specifically contemplated unless specifically indicated to the contrary. Likewise, any subset or combination of these is also specifically contemplated and disclosed. This concept applies to all aspects of this disclosure. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

### EXAMPLES

### Example 1

### Method of Reducing GC Bias Using Patterned Surface

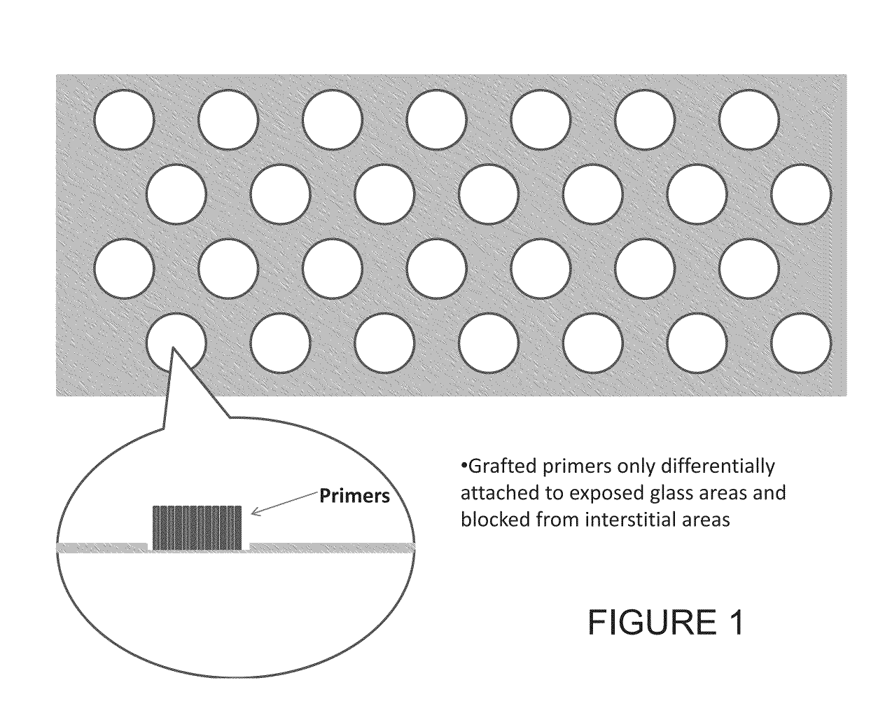

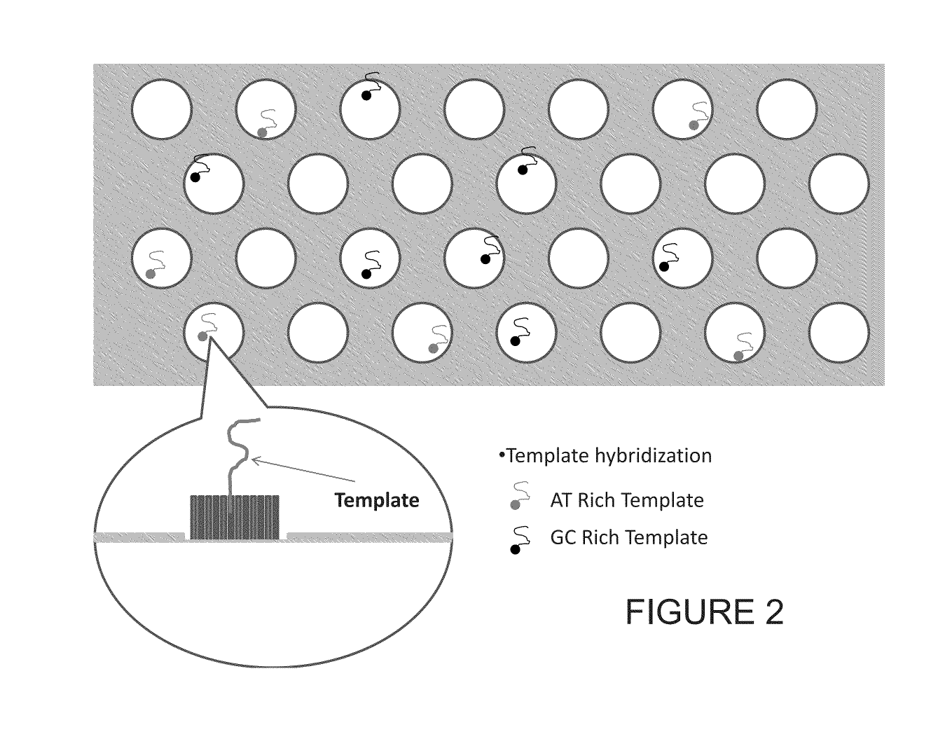

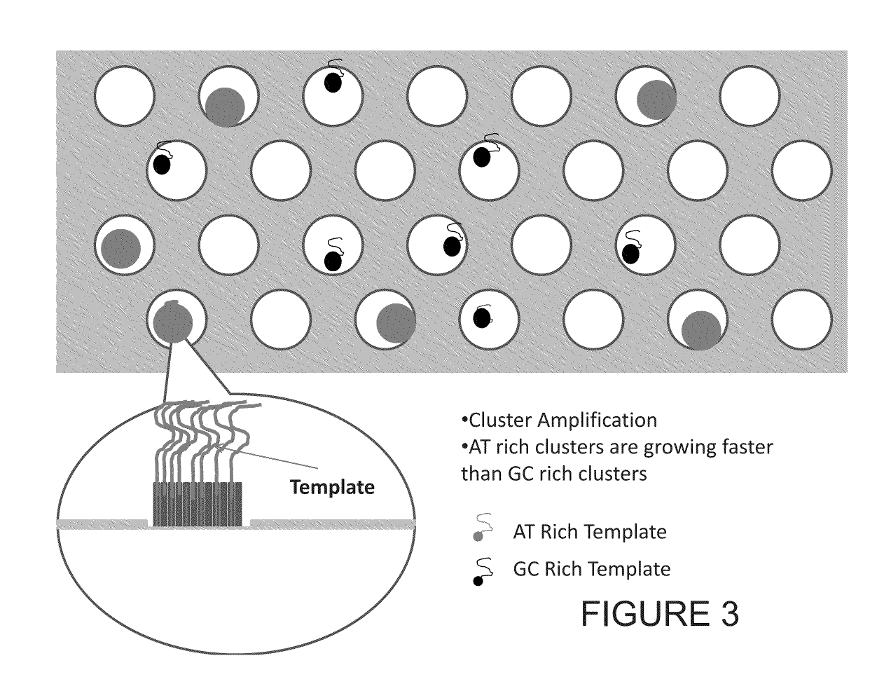

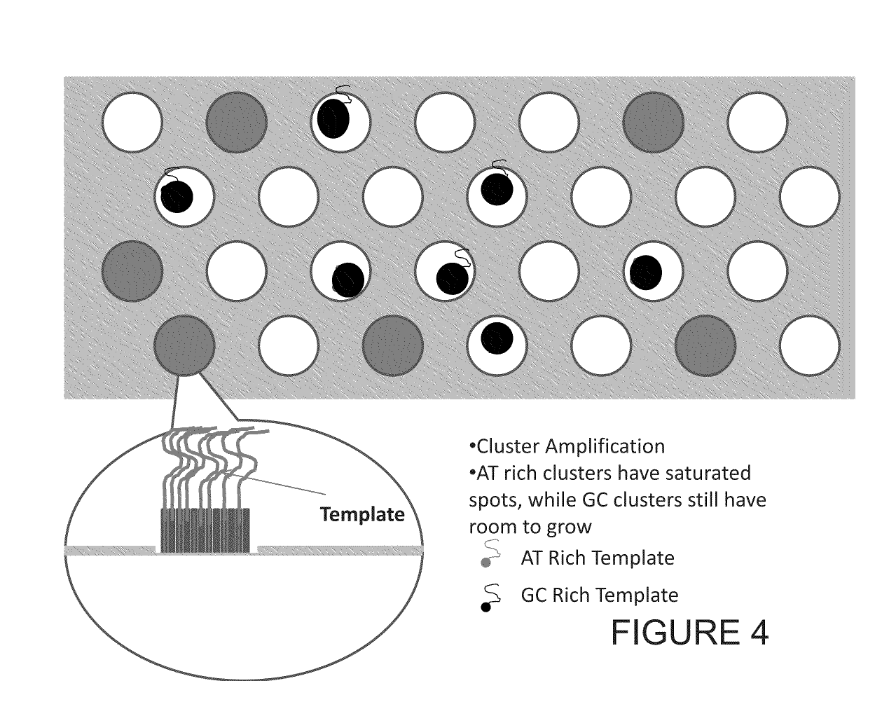

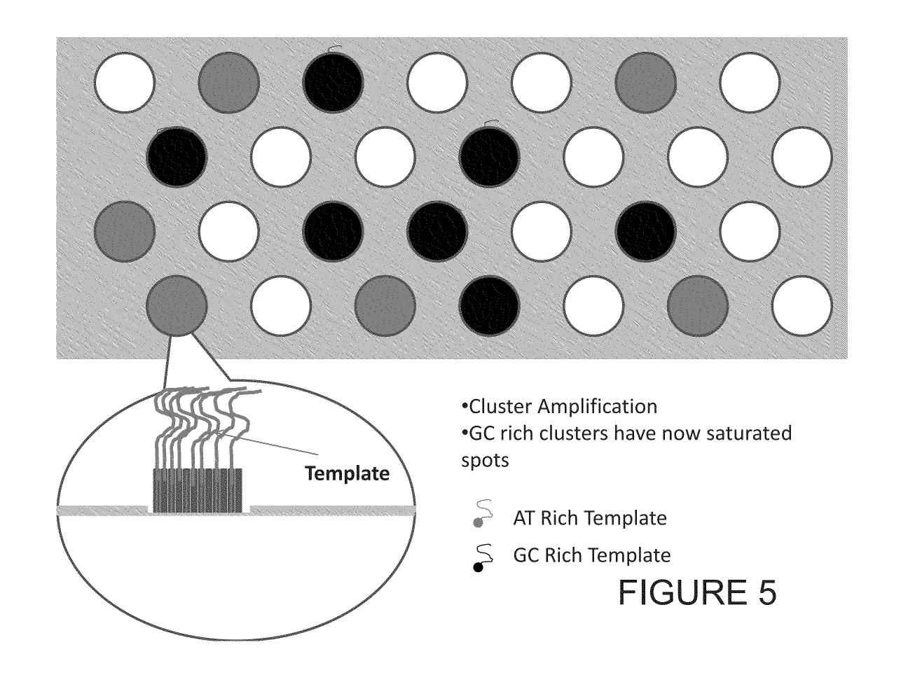

In standard clustering, template nucleic acid molecules randomly seed across an entire surface covered by primers. As shown in FIG. 1, using a patterned surface, such as a patterned flowcell, template nucleic acid molecules seed only isolated patches of primers. These primer patches can end up with zero, one or multiple template nucleic acid molecules. However, as shown in FIG. 2, a concentration of template nucleic acid molecules is used that allow distribution of template nucleic acid molecules across the surface such that primer patches usually contain one template. A typical Poisson distribution would predict about 35-37% of the primer patches to have one template. After initial extension, the nucleic acid molecules are amplified. As shown in FIG. 3, GC rich template clusters grow slower than AT rich template clusters. With typical surfaces entirely covered by primers, GC rich cluster growth could be stunted by competing AT rich clusters that deplete usable primers. As shown in FIGS. 1-5, using patterned flowcells, AT rich clusters are isolated from neighboring clusters. Thus, clusters, whether AT or GC rich, are allowed to grow or amplify until the primers in a patch are saturated. See FIGS. 3-5. The evenly sized clusters can now be analyzed, e.g., sequenced, without a bias due to size.

### Example 2

### Method of Reducing GC Bias By Repeated Loading and Continuous Amplification Using a Patterned Surface and a Recombinase Agent

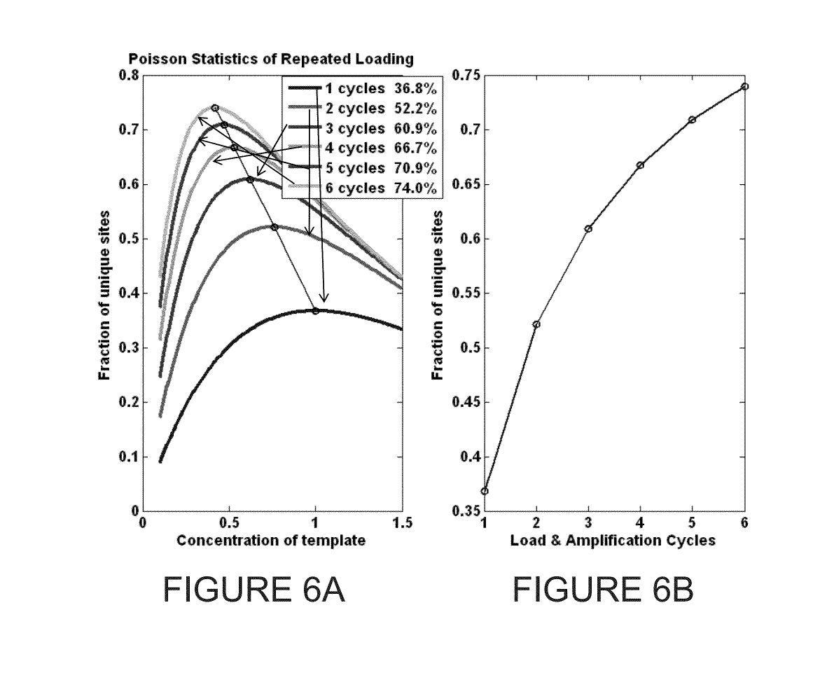

A tube sufficiently long to hold the required volume of all reagents required for loading and amplification (i.e., nucleic acid molecule templates to be amplified; buffers; amplification solutions; etc.) are loaded sequentially into a tube leading to a surface, such as a flowcell, comprising patches of primers. The reagents are then pumped forward with appropriate timing to achieve saturated amplification of all patches having nucleic acid molecule templates. Flow is then reversed such that the solution comprising the nucleic acid molecule templates is brought back to the surface for subsequent loading of the templates onto primer patches. Forward flow is repeated to amplify the new templates to saturation. As shown in FIG. 6A, six cycles of this process would result in an approximate doubling of uniquely populated patches. This process is carried out in the presence of a recombinase agent, as described above, to simplify the amplification method by carrying out amplification without thermal melting.

A number of embodiments have been described. Nevertheless, it will be understood that various modifications may be made. Accordingly, other embodiments are within the scope of the following claims.

1. A method for amplifying nucleic acid molecules of different sequence comprising:
a. applying to a plurality of patches of primers immobilized on a solid surface, a first solution comprising a plurality of nucleic acid molecules of different sequence under conditions wherein one or more of the nucleic acid molecules anneals to one or more primers in a patch of primers and the annealed nucleic acid molecules are amplified until the patch is saturated to produce colonies of immobilized nucleic acid molecules;
b. applying to the patches of immobilized primers and colonies of step (a), a second solution comprising a plurality of nucleic acid molecules of different sequence under conditions wherein one or more of the nucleic acid molecules anneals to one or more primers in a patch of primers and the annealed nucleic acid molecules are amplified until the patch is saturated to produce colonies of immobilized nucleic acid molecules.

2. The method of claim 1, wherein one nucleic acid molecule anneals to one patch of primers.

3. (canceled)

4. The method of claim 1, wherein the annealed nucleic acid molecules are continuously amplified until the primers in a patch are saturated.

5. The method of claim 1, wherein the first solution is applied in a first direction and the second solution is applied in a second direction.

6. The method of claim 2, wherein the first solution is the same as the second solution.

7. The method of claim 6, wherein the solution is applied in the second direction by changing the direction of flow of the solution.

8. The method of claim 2, wherein the first direction is opposite to the second direction.

9. The method of claim 2, wherein the second solution is different from the first solution.

10. (canceled)

11. The method of claim 1, wherein step (b) is repeated.

12-15. (canceled)

16. The method of claim 1, further comprising a finishing step wherein the concentration of the plurality of nucleic acid molecules is higher than the concentration of the plurality of nucleic acid molecules in the previous step.

17. The method of claim 1, wherein the nucleic acid molecules are amplified under isothermal conditions.

18. The method of claim 1, wherein the nucleic acid molecules are amplified under thermal conditions.

19. The method of claim 1, wherein each colony comprises nucleic acid molecules of the same sequences.

20. The method of claim 1, wherein the sequence of the nucleic acid molecules of one colony is different from the sequence of the nucleic acid molecules of another colony.

21. The method of claim 1, wherein each colony comprises a different nucleic acid sequence.

22. The method of claim 1, wherein the first or second solution comprises a recombinase agent.

23-25. (canceled)

26. The method of claim 1, wherein the colonies are of approximately equal size.

27. (canceled)

28. (canceled)

29. A method of solid phase amplification comprising
a. providing a surface comprising a plurality of patches of primers;
b. providing a plurality of different nucleic acid molecules;
c. contacting the plurality of different nucleic acid molecules with the surface under conditions wherein the nucleic acid molecules bind to primers at only a subset of the patches;
d. amplifying the nucleic acid molecules under conditions to saturate the subset of patches with copies of the nucleic acid molecules; and
e. repeating steps (c) and (d), thereby increasing the number of patches that are saturated with copies of the nucleic acid molecules.

30. The method of claim 29, wherein the same primer sequences are present at the patches in the plurality of patches.

31. The method of claim 29, wherein nucleic acid molecules in the plurality of different nucleic acid molecules comprise common primer binding sequences flanking different target sequences.

32. (canceled)

33. The method of claim 32, wherein the subset of patches that saturate with copies of nucleic acid molecules comprise patches having copies of AT rich sequences and patches having copies of the GC rich sequences.

34. The method of claim 33, wherein the patches having copies of the AT rich sequences are of approximately equal density as the patches having copies of the GC rich sequences.

35. The method of claim 33, wherein the patches having copies of the AT rich sequences are of approximately equal copy number as the patches having copies of the GC rich sequences.

36. (canceled)

37. The method of claim 29, wherein the conditions under which the plurality of different nucleic acid molecules is contacted with the surface produces a Poisson distribution of occupancy in the plurality of patches that are bound to nucleic acids in the plurality of different nucleic acid molecules.

38. (canceled)
